(19) United States
(12) Patent Application Publication
(10) Pub. No.: US 2021/0128895 A1
(43) Pub. Date: May 6, 2021

(54) DEVICE

(71) Applicant: AustarPharma, LLC, Edison, NJ (US)

(72) Inventors: Weiqin TONG, Edison, NJ (US); Xiaohong QI, Edison, NJ (US)

(21) Appl. No.: 17/085,341

(22) Filed: Oct. 30, 2020

Related U.S. Application Data
(60) Provisional application No. 62/928,088, filed on Oct. 30, 2019.

(51) Int. Cl. A61M 35/00; A61K 9/08; A61K 31/485

(52) U.S. Cl. CPC A61M 35/003; A61K 31/485; A61K 9/08

(57) ABSTRACT

Disclosed is a drug delivery device comprising a body for holding a first solution and a second solution which are independent with each other; a pipeline delivery system comprising one or more pipelines for independently delivering the first solution and the second solution, respectively; a press device for sucking the first solution and the second solution into the pipelines, and a nozzle for delivering the first solution and the second solution in the pipelines to an administration site such that the first solution covers the second solution at the administration site.

### CROSS-REFERENCE TO RELATED APPLICATIONS

The present application claims priority to and the benefit of U.S. Provisional Application No. 62/928,088, filed Oct. 30, 2019, the disclosure of which is hereby incorporated by reference in its entirety.

### FIELD

The present disclosure generally relates to the medicinal field. In particular, the present disclosure relates to the field of medical devices.

### BACKGROUND

The liquid drug delivery device of the press spray type is mostly a single-chamber drug delivery device, that is, one drug delivery device has only one liquid storage chamber, and only one liquid can be applied in one administration.

### SUMMARY

In one aspect, the present disclosure relates to a drug delivery device comprising a body for holding a first solution and a second solution which are independent with each other; a pipeline delivery system comprising one or more pipelines for independently delivering the first solution and the second solution respectively; a press device for sucking the first solution and the second solution into the pipelines, and a nozzle for delivering the first solution and the second solution in the pipelines to an administration site such that the first solution covers the second solution at the administration site.

In some embodiments, the body comprises a plurality of containers for independently holding the first solution and the second solution.

In some embodiments, the body comprises a plurality of regions for independently holding the first solution and the second solution.

In some embodiments, the pipeline comprises a suction portion and a delivery portion.

In some embodiments, the suction portions of the pipelines are independent of each other.

In some embodiments, the delivery portions of the pipelines are independent of each other.

In some embodiments, the delivery portions of the pipeline are in contact with each other.

In some embodiments, the press device comprises a pump and a tube.

In some embodiments, the first solution and the second solution reach the administration site successively from the nozzles.

In some embodiments, the first solution and the second solution reach the administration site simultaneously.

In some embodiments, the nozzle has one or more channels.

In some embodiments, the nozzle has a first channel and a second channel, and the first channel and the second channel are independent with each other.

In some embodiments, the nozzle has a first channel and a second channel, and the first channel is concentric with the second channel.

In some embodiments, the sections of the first channel and the second channel are all circular.

In some embodiments, the nozzle has a first channel and a second channel, the first channel is located in the second channel, and the cross-sectional release area of the first channel is greater than the cross-sectional release area of the second channel.

In another aspect, the present disclosure relates a method for improving the stability of a drug comprising administering the drug to a subject with a drug delivery device, wherein the drug delivery device comprises a body for holding a first solution and a second solution which are independent with each other; a pipeline delivery system comprising one or more pipelines for independently delivering the first solution and the second solution, respectively; a press device for sucking the first solution and the second solution into the pipelines, and a nozzle for delivering the first solution and the second solution in the pipelines to an administration site such that the first solution covers the second solution at the administration site.

In some embodiments, the drug is apomorphine or a pharmaceutically acceptable salt thereof.

In some embodiments, the first solution comprises the drug.

In some embodiments, the second solution comprises an alkaline polymer solution.

### BRIEF DESCRIPTION OF THE DRAWINGS

FIG. 1 is a schematic diagram of the drug delivery device of some embodiments of the present disclosure, wherein FIG. 1(1) is a schematic diagram of the drug delivery device of one of the embodiments; and FIG. 1(2) is a schematic diagram of the drug delivery device of the other of the embodiments.

FIG. 2 is a schematic diagram of the drug delivery device of some embodiments of the present disclosure; FIG. 2(1) is a cross-sectional structural schematic diagram of the drug delivery device; FIG. 2(2) and FIG. 2(3) are structural schematic diagrams of the pump pipeline f1 and the pump pipeline f2.

FIG. 3 is a schematic diagram of the drug delivery device of some embodiments of the present disclosure.

FIG. 4 is a schematic diagram of the drug delivery device of some embodiments of the present disclosure; FIG. 4(1) is a cross-sectional structural schematic diagram of the drug delivery device; FIG. 4(2) and FIG. 4(3) are structural schematic diagrams of the pump pipeline f1 and the pump pipeline f2.

FIG. 5 is a structural schematic diagram of a first sucking mechanism in the drug delivery device of some embodiments of the present disclosure.

FIG. 6 is an overall schematic diagram of the drug delivery device of some embodiments of the present disclosure.

FIG. 7 is an overall schematic diagram of the drug delivery device of some embodiments of the present disclosure.

FIG. 8 is a schematic diagram of the drug delivery device of some embodiments of the present disclosure.

FIG. 9 is a partial enlarged diagram of the drug delivery device shown in FIG. 8.

FIG. 10 is a schematic diagram of the drug delivery device of some embodiments of the present disclosure.

FIG. 11 is a partial enlarged diagram of the drug delivery device shown in FIG. 10.

FIG. 12 is a schematic diagram of the pump pipeline f1 and the pump pipeline f2 in some embodiments; FIG. 12(1) shows the pump pipeline f1 and the pump pipeline f2 separated by separator I; FIG. 12(2) shows that the pump pipeline f1 and the pump pipeline f2 separated by a round of separator J.

FIG. 13 is a schematic diagram showing a multifunctional nozzle in some embodiments; FIG. 13(1) shows that the first liquid outlet is provided at the periphery of the second liquid outlet; FIG. 13(2) shows that the first liquid outlet is connected to the second liquid outlet; FIG. 13(3) shows the independent first liquid outlet and second liquid outlet.

FIG. 14 is a schematic diagram of the drug delivery device of some embodiments of the present disclosure.

FIG. 15 is a schematic diagram of the drug delivery device of some embodiments of the present disclosure.

FIG. 16 is a partial enlarged diagram of the drug delivery device shown in FIG. 15.

FIG. 17 is a schematic diagram of the drug delivery device of some embodiments of the present disclosure.

FIG. 18 is a partial enlarged diagram of the drug delivery device shown in FIG. 17.

FIG. 19 is a schematic diagram of the drug delivery device of some embodiments of the present disclosure.

FIG. 20 is a partial enlarged diagram of the drug delivery device shown in FIG. 19.

FIG. 21 is a schematic diagram of the drug delivery device of some embodiments of the present disclosure.

FIG. 22 is a partial enlarged diagram of the drug delivery device shown in FIG. 21.

FIG. 23 is a schematic diagram of the drug delivery device of some embodiments of the present disclosure.

### DETAILED DESCRIPTION

In the following description, some specific details are included to provide a comprehensive understanding of the various disclosed embodiments. However, one skilled in the relevant art will recognize that the embodiments may be practiced without the use of one or more of these specific details and other methods, components, materials and the like.

Unless specified otherwise, the words “include”, “comprise”, “contain” and “have” are to be interpreted as an open, inclusive meaning, i.e. “including but not limited to” in the entire specification and the appended claims.

References to “one embodiment”, “an embodiment”, “in another embodiment” or “in some embodiments” throughout the specification are meant to include in the at least one embodiment relevant specific reference elements, structures or features. The appearances of the phrase “in one embodiment” or “in an embodiment” or “in another embodiment” or “in some embodiments” are not necessarily all referring to the same embodiment. Furthermore, the particular elements, structures, or characteristics may be combined in any suitable manner in one or more embodiments.

It should be noted that, as used in this specification and the appended claims, the singular forms “a”, “an” and “the” include plural referents unless the context clearly stated otherwise. Therefore, for example, a reaction comprising “a pharmaceutically acceptable excipient” comprises one pharmaceutically acceptable excipient, two or more pharmaceutically acceptable excipients.

### Definition

As used herein, the term “pharmaceutically acceptable salts” includes “acceptable acid addition salts” and “acceptable base addition salts”.

As used herein, the term “acceptable acid addition salt” refers to those salts which retain the biological effectiveness and properties of the free base, which are not biologically or otherwise undesirable, and which are formed with inorganic acids or organic acids. The inorganic acid is, for example but not limited to, hydrochloric acid, hydrobromic acid, sulfuric acid, nitric acid, phosphoric acid and the like. The organic acid is, for example, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

As used herein, the term “acceptable base addition salt” refers to those salts which retain the biological effectiveness and properties of the free acid, which are not biologically or otherwise undesirable. These salts are prepared from addition of an inorganic base or an organic base to the free acid. Salts derived from inorganic bases include, but not limited to, the sodium, potassium, lithium, ammonium, calcium, magnesium, iron, zinc, copper, manganese, aluminum salts and the like. In some embodiments, the inorganic salts are ammonium, sodium, potassium, calcium and magnesium salts. Salts derived from organic bases include, but not limited to salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purine, piperazine, piperidine, N-ethylpiperidine, polyamine resins and the like. In some embodiments, the organic base is isopropylamine, diethylamine, ethanolamine, trimethylamine, dicyclohexylamine, choline and caffeine.

As used herein, the term “release area” refers to the area of the cross section of the channel through which the solution can pass.

### Specific Embodiments

In one aspect, the present disclosure relates to a drug delivery device, comprising

a body for holding a first solution and a second solution which are independent with each other;

a pipeline delivery system comprising one or more pipelines for independently delivering the first solution and the second solution respectively;

a press device for sucking the first solution and the second solution into the pipelines, and

a nozzle for delivering the first solution and the second solution in the pipelines to an administration site such that the first solution covers the second solution at the administration site.

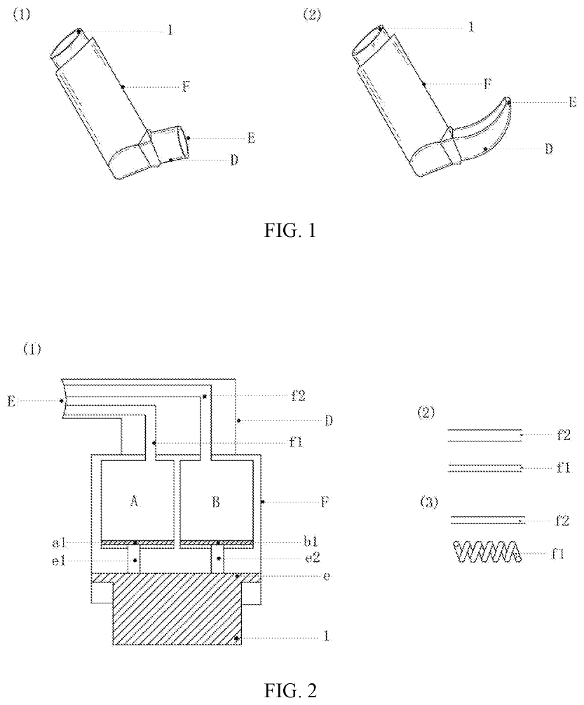

Referring to FIGS. 1 to 3, the drug delivery device comprises a press device 1 (a press portion e is provided on the press device 1), a pipeline delivery system, a body F and a nozzle E.

In some embodiments, the drug delivery device further comprises a pump-out section for pumping out the first solution in the first chamber A and the second liquid in the second chamber B, respectively. The pump-out section comprises pump-out pipelines f1 and f2 for delivering the first solution in the first chamber A and the second solution in the second chamber B, respectively.

The press portion e is connected, respectively, with the pistons (the first chamber press piston a1 and the second chamber press piston b1) located in the first chamber A and the second chamber B through the two connecting rods e1 and e2.

Since the press portion e is connected, respectively, with the first chamber press piston a1 and the second chamber press piston b1 through the two connecting rods e1 and e2, when the press portion e is pressed, the first chamber press piston a1 and the second chamber press piston b1 is, respectively, subject to pressure to move in the first chamber A and the second chamber B. Therefore, the volumes of the first chamber A and the second chamber B are reduced, while the pressure is increased, thereby making the first solution in the the first chamber A and the second solution in the second chamber B flow out from the pump-out pipeline f1 of the first chamber A and the pump-out pipeline f2 of the second chamber B, respectively.

In the present exemplary embodiment, whether the second solution in the second chamber B at the administration site covers the first solution in the first chamber A depends on the setting of the pump-out pipeline f1 and the pump-out pipeline f2, which comprises the following two scenarios:

(1) The pump-out pipeline f1 and the pump-out pipeline f2 are pipelines with the same length but different cross-sectional areas (as shown in FIG. 2(2)). Therefore, after one press, the time during which first solution in the first chamber A and the second solution in the second chamber B flow out of the press device is different, thereby achieving the purpose of delivering different solutions one after another after one press.

(2) The pump-out pipeline f1 and the pump-out pipeline f2 are pipelines with the same cross-sectional area but different lengths (as shown in FIG. 2(3), for example, one is a linear pipeline and the other is a spirally curved pipeline). Therefore, after one press, the time during which the first solution in the first chamber A and the second solution in the second chamber B flow out of the press device is different, thereby also achieving the purpose of delivering different solutions one after another after one press.

The dual-chamber drug delivery device as described above can be easily modified for sequential delivery of different solutions in three chambers, four chambers, or even more chambers. For example, the press cover e is provided with four connecting rods, wherein the first connecting rod inserts into the first chamber and is connected with the first press piston in the first chamber, and the first chamber is provided with a first pump-out pipeline for delivering the solution outwards; the second connecting rod inserts into the second chamber and is connected with the second press piston in the second chamber, and the second chamber is provided with a second pump-out pipeline for delivering the solution outwards; the third connecting rod inserts into the third chamber and is connected with the third press piston in the third chamber, and the third chamber is provided with a third pump-out pipeline for delivering the solution outwards; the fourth connecting rod inserts into the fourth chamber and is connected with the fourth press piston in the fourth chamber, and the fourth chamber is provided with a fourth pump-out pipeline for delivering the solution outwards.

The above-described multiple chamber drug delivery device operates on the same principle as the dual-chamber drug delivery device.

The drug delivery device can further comprise an output portion D for accommodating the pump-out pipeline f1 and the pump-out pipeline f2, and the output portion D is provided with a nozzle F.

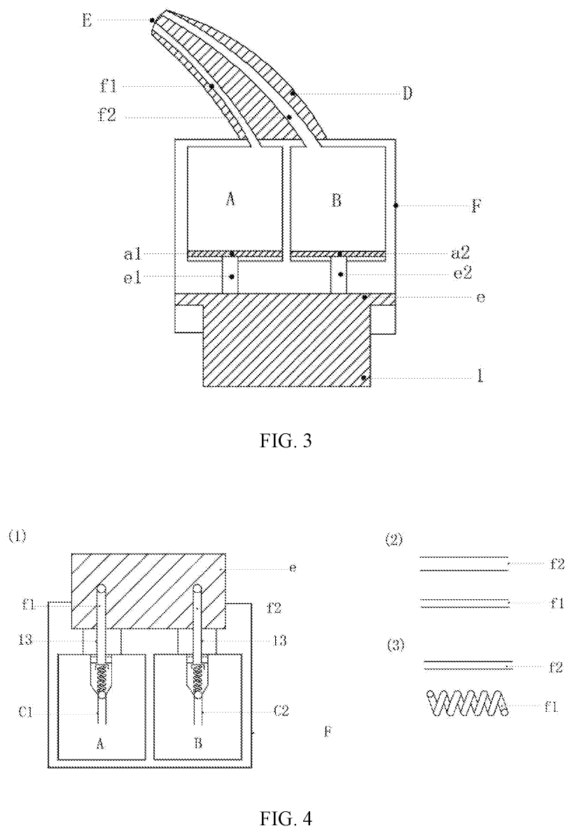

In some embodiments, when the drug delivery device is used in a place where the administration site is small or where the administration site may be in contact with a small area (for example, at the inner wall of the nasal cavity, at the nasal limen or in the oral cavity), the output portion D may be a columnar body whose cross-sectional area gradually decreases toward the solution outlet position (as shown in FIG. 3).

In some embodiments, if it is desired to obtain a hazy solution, a device for atomizing the solutions delivered respectively from the pump-out pipeline f1 and the pump-out pipeline f2 can be provided outside the liquid outlets of the above-described drug delivery device.

In some embodiments, the drug delivery device can be a disposable device, i.e., disposable after a single administration.

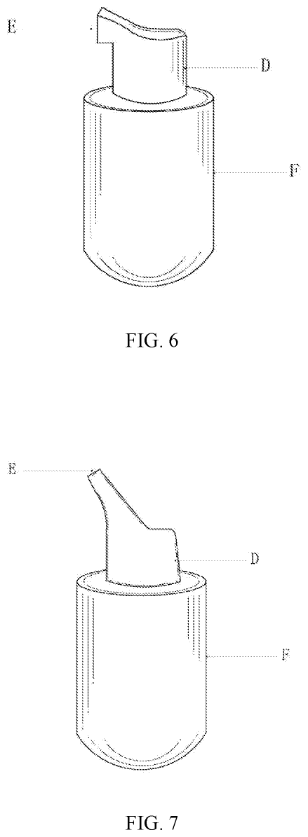

Referring to FIGS. 4 to 7, the drug delivery device comprises a press device 1 (a press portion e is provided on the press device 1), a pipeline delivery system, a body F and a nozzle E.

In some embodiments, the drug delivery device further comprises a fluid-sucking portion C for sucking the first solution in the first chamber A and the second solution in the second chamber B, respectively (the fluid-sucking portion of the first chamber A is referred to as C1, the fluid-sucking portion of the second chamber B is referred to as C2), the first chamber fluid-sucking mechanism, the second chamber fluid-sucking mechanism and a pump-out portion for pumping out the sucked solution.

In the present exemplary embodiment, the first chamber A and the second chamber B are each formed by separate containers. In some embodiments, the first chamber A and the second chamber B may also be two separate chambers formed in the same container and separated by a separator. In some embodiments, the first chamber A and the second chamber B may also be two separate containers that are disposed in the same container but are in contact with one another.

The structures of the first chamber fluid-sucking mechanism and the second chamber fluid-sucking mechanism are fully identical. The fluid-sucking mechanism used in the present embodiment is a conventional fluid-sucking mechanism in this filed, which is a power device for sucking liquid in each chamber. In order to more clearly describe the structure of the present embodiment, one specific structure of a fluid-sucking mechanism is described herein. However, other fluid-sucking mechanisms having the same functions as those of the fluid-sucking mechanism described in the present embodiment can be also used in the present disclosure.

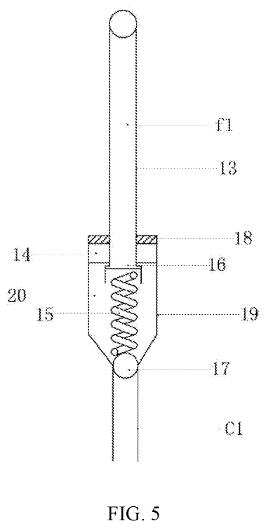

Taking the fluid-sucking mechanism in the first chamber A (such as the first chamber fluid-sucking mechanism as shown in FIG. 5) as an example, the fluid-sucking mechanism comprises: a cover 18 disposed at the outlet of the chamber, of which the side extends into the chamber to form a cover inner wall 19 having the tubular structure; a gland connecting shaft 13 connected with the press cover e and extending into the chamber, in which the gland connecting shaft 13 has a hollow structure, one end of which extends into the chamber and the other end leads to the outside of the press cover e, thereby discharging the fluid (the pipelines formed by the hollow portion in the gland connecting shafts of the first chamber A and the second chamber B are referred to as the pump-out pipeline f1 and the pump-out pipeline f2, respectively); a press piston 14 disposed in the chamber and surrounding the gland connecting shaft 13, which abuts against the cover inner wall 19; a press spring 15 connected with the underside of the press piston 14, in which the upper end of the press spring 15 is provided with an exhaust valve 16, the lower end of the press spring 15 is provided with an inlet valve 17 (an exemplary example of the inlet valve 17 in the present embodiment is a circular plastic ball provided on the spring) such that a reservoir 20 is formed between the exhaust valve 16 and the inlet valve 17.

The working principle of the first chamber fluid-sucking mechanism is as follows: when the press cover e is pressed downward, the gland connecting shaft 13 moves downward and at the same time drives the press piston 14 to move downward. At this moment, the exhaust valve 16 is opened, and the press spring 15 is compressed downward to close the inlet valve 17. When the press cover e is released, the press spring 15 is reset such that the inlet valve 17 is opened and the exhaust valve 16 is closed. The press piston 14 is moved upward, so that the volume of the reservoir 20 becomes larger, and the pressure becomes smaller. Therefore, the first solution in the chamber A enters the reservoir 20 through the fluid-sucking portion C1. When the press cover e is pressed down again, the volume inside the reservoir 20 becomes smaller and the pressure becomes larger, thereby causing the solution in the reservoir 20 to flow out through the pump-out pipeline f1.

It can be seen that the working principle of the drug delivery device of the present exemplary embodiment is as follows: when pressing the press cover e to cause the first chamber fluid-sucking mechanism and the second chamber fluid-sucking mechanism to operate, the first solution in the first chamber A and the second solution in the second chamber B enter the reservoir of the first chamber fluid-sucking mechanism and the reservoir of the second chamber fluid-sucking mechanism through the fluid-sucking portions C1 and C2, respectively, and then are delivered through the pump-out pipeline f1 and the pump-out pipeline f2, respectively.

In the present exemplary embodiment, whether the second solution in the second chamber B at the administration site covers the first solution in the first chamber A depends on how to combine the settings of the fluid-sucking portion C1, the fluid-sucking portion C2, the pump-out pipeline f1 and the pump-out pipeline f2, which comprises the following six scenarios:

(1) The fluid-sucking portion C1 and the fluid-sucking portion C2 are completely identical. The pump-out pipeline f1 and the pump-out pipeline f2 are pipelines with the same length but different cross-sectional areas (as shown in FIG. 4(2)). Therefore, after one press, the time required for the first solution in the first chamber A to pass through the fluid-sucking portion C1 and the pump-out pipeline f1 and the time required for the second solution in the second chamber B to pass through the fluid-sucking portion C2 and the pump-out pipeline f2 are different, thereby achieving the purpose of delivering different solutions one after another after one press.

(2) The fluid-sucking portion C1 and the fluid-sucking portion C2 are completely identical. The pump-out pipeline f1 and the pump-out pipeline f2 are pipelines with the same cross-sectional area but different lengths (as shown in FIG. 4(3), for example, one is a linear pipeline and the other is a spirally curved pipeline). Therefore, after one press, the time required for the first solution in the first chamber A to pass through the fluid-sucking portion C1 and the pump-out pipeline f1 and the time required for the second solution in the second chamber B to pass through the fluid-sucking portion C2 and the pump-out pipeline f2 are different, thereby achieving the purpose of delivering different solutions one after another after one press.

(3) The fluid-sucking portion C1 and the pump-out pipeline f1 are completely identical (herein, the fluid-sucking portion C1 and the pump-out pipeline f1 are collectively referred to as the first chamber delivery pipeline). The fluid-sucking portion C2 and the pump-out pipeline f2 are completely the same (herein, the fluid-sucking portion C2 and the pump-out pipeline f2 are collectively referred to as the second chamber delivery pipeline). However, the lengths of the first chamber delivery pipeline and the second chamber delivery pipeline are the same, but the cross-sectional areas thereof are different (as shown in FIG. 4(2)). Therefore, after one press, the time required for the first solution in the first chamber A to pass through first chamber delivery pipeline and the time required for the second solution in the second chamber B to pass through the second chamber delivery pipeline are different, thereby achieving the purpose of delivering different solutions one after another after one press.

(4) The fluid-sucking portion C1 and the pump-out pipeline f1 are completely identical (herein, the fluid-sucking portion C1 and the pump-out pipeline f1 are collectively referred to as the first chamber delivery pipeline). The fluid-sucking portion C2 and the pump-out pipeline f2 are completely the same (herein, the fluid-sucking portion C2 and the pump-out pipeline f2 are collectively referred to as the second chamber delivery pipeline). However, first chamber delivery pipeline and the second chamber delivery pipeline have the same cross-sectional area but different lengths (as shown in FIG. 4(3), for example, one is a linear pipeline and the other is a spirally curved pipeline). Therefore, after one press, the time required for the first solution in the first chamber A to pass through first chamber delivery pipeline and the time required for the second solution in the second chamber B to pass through the second chamber delivery pipeline are different, thereby achieving the purpose of delivering different solutions one after another after one press.

(5) The fluid-sucking portion C1 and the pump-out pipeline f2 (or the fluid-sucking portion C2 and the pump-out pipeline f1) are completely identical. The fluid-sucking portion C2 and the pump-out pipeline f1 (or the fluid-sucking portion C1 and the pump-out pipeline f2)) are pipelines with the same cross-sectional area but different lengths (as shown in FIG. 4(3), for example, one is a linear pipeline and the other is a spirally curved pipeline). Therefore, after one press, the time required for the first solution in the first chamber A to pass through first chamber delivery pipeline and the time required for the second solution in the second chamber B to pass through the second chamber delivery pipeline are different, thereby achieving the purpose of delivering different solutions one after another after one press.

(6) The fluid-sucking portion C1 and the pump-out pipeline f2 (or the fluid-sucking portion C2 and the pump-out pipeline f1) are completely identical. The fluid-sucking portion C2 and the pump-out pipeline f1 (or the fluid-sucking portion C1 and the pump-out pipeline f2)) are pipelines with the same length but different cross-sectional areas (as shown in FIG. 4(2)). Therefore, after one press, the time required for the first solution in the first chamber A to pass through first chamber delivery pipeline and the time required for the second solution in the second chamber B to pass through the second chamber delivery pipeline are different, thereby achieving the purpose of delivering different solutions one after another after one press.

The dual-chamber drug delivery device as described above can be extended to a three-chamber, four-chamber, or even more chamber drug delivery device.

In some embodiments, when the drug delivery device is used in a place where the administration site is small or where the administration site may be in contact with a small area (for example, at the inner wall of the nasal cavity, at the nasal limen or in the oral cavity), the output portion D may be a columnar body whose cross-sectional area gradually decreases toward the solution outlet position (as shown in FIG. 7).

In some embodiments, if it is desired to obtain a hazy solution, a device for atomizing the solutions delivered respectively from the pump-out pipeline f1 and the pump-out pipeline f2 can be provided outside the liquid outlets of the above-described drug delivery device.

In some embodiments, the drug delivery device can be a disposable device, i.e., disposable after a single administration.

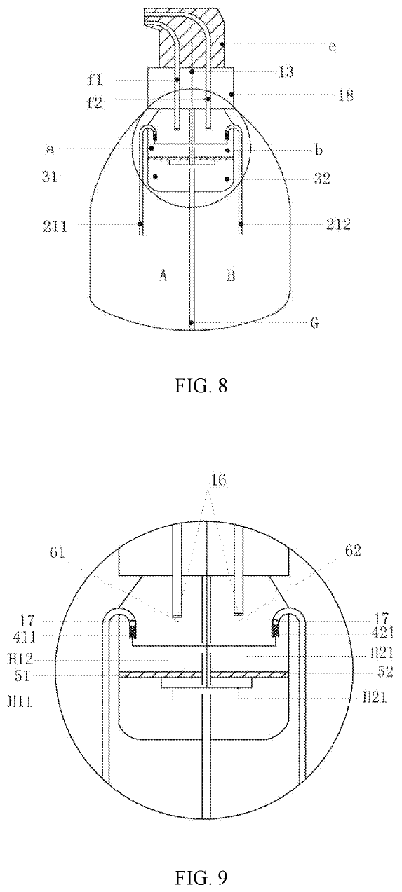

Referring to FIGS. 8 to 14, the drug delivery device comprises a press device 1 (a press portion e is provided on the press device 1), a pipeline delivery system, a body F and a nozzle E.

For convenience of description, in the present embodiment, the first chamber A and the second chamber B are formed by a separator G fixedly disposed in the same body, wherein the separator G is provided with a hollow portion and extends from the bottle mouth of the body to the bottom of the body.

The press portion 1 comprises a press cover e, a cover 18 provided on the outlet of the body of the bottle, pump pipelines disposed inside the press cover e (i.e., the pump-out portion. In the present embodiment, there are the first chamber pump-out pipeline (pump-out pipeline f1) and the second chamber pump-out pipeline (pump-out pipeline f2)). In the present embodiment, the pump-out pipeline f1 and the pump-out pipeline f2 deliver the same amount of fluid in the same length of pipeline per unit time. The lower part of the press cover e is connected with the gland connecting shaft 13. The gland connecting shaft 13 is disposed inside the separator G of the hollow portion.

The drug delivery device comprises: a fluid-sucking portion 211 and a fluid-sucking portion 212 for respectively sucking the first solution in the first chamber A and the second solution in the second chamber B; a first temporary reservoir cavity 31 and a second temporary reservoir cavity 32 for respectively storing the solution delivered from the first chamber A and the second chamber B through the fluid-sucking portions (the first temporary reservoir cavity 31 and the second temporary reservoir cavity 32 are respectively provided with a liquid pump outlet 61 of the first temporary reservoir cavity and a liquid pump outlet 62 of the second temporary reservoir cavity, and the liquid pump outlet 61 of the first temporary reservoir cavity and the liquid pump outlet 62 of the second temporary reservoir cavity lead to the pump-out pipeline f1 and the pump-out pipeline f2, respectively); wherein the first temporary reservoir cavity 31 and the second temporary reservoir cavity 32 are separated by a separator G of the hollow portion.

The outlets of the fluid-sucking portion 211 and the fluid-sucking portion 212 are respectively provided with a first spring device and a second spring device. In the first temporary reservoir cavity 31 and the second temporary reservoir cavity 32, a first separator 51 and a second separator 52 which can raise or lower are provided in the first temporary reservoir cavity 31 and the second temporary reservoir cavity 32, respectively.

In the present embodiment, the first spring device and the second spring device have identical structure, which comprises a spring and an inlet valve 17 provided at the outlet end of the fluid-sucking portion (an exemplary example of the inlet valve in the present embodiment is a circular plastic ball arranged on a spring). That is, the first spring device comprises a first chamber spring 411 and an inlet valve 171 at the outlet end of the fluid-sucking portion 211 and the second spring device comprises a second chamber spring 421 and an inlet valve 172 at the outlet end of the fluid-sucking portion 212.

The gland connecting shaft 13 is provided with four connecting legs which extend from the separator G of the hollow portion and extend to the first temporary reservoir cavity 31 and the second temporary reservoir cavity 32, respectively. The two connecting legs H11 and H12 in the the first temporary reservoir cavity 31 are connected with the first separator 51 and the first chamber spring 411, respectively. The two connecting legs H21 and H22 in the second temporary reservoir cavity 32 are connected with the second separator 52 and the second chamber spring 421, respectively (Certainly, the two connecting legs H11 and H12 in the the first temporary reservoir cavity 31 can be connected with the first chamber spring 411 and the first separator 51, respectively. The two connecting legs H21 and H22 in the second temporary reservoir cavity 32 can be connected with the second chamber spring 421 and the second partition 52, respectively).

In the present embodiment, the first separator 51 and the second separator 52 divide the first temporary reservoir cavity 31 and the second temporary reservoir cavity 32 into a part of the cavity close to the press cover e and a part of the cavity away from the press cover e, respectively. The outlet ends of the fluid-sucking portion 211 and the fluid-sucking portion 212 are located in a part of the cavity close to the press cover e, respectively (for the sake of clarity, the bottle mouth of the body is upward. The outlet ends of the fluid-sucking portion 211 and the fluid-sucking portion 212 are located above the first separator 51 and the second separator 52, respectively, wherein the upper portions of the first separator 51 and the second separator 52 are the cavity a and the cavity b, respectively). The working principle of the drug delivery device is as follows: the press cover e is moved downward by the external pressure such that the gland connecting shaft 13 is simultaneously moved downward. As the connecting legs of the gland connecting shaft 13 are connected with the spring and the separator, respectively, in each chamber, when the gland connecting shaft moves downward, the spring is stretched. On one hand, when the first chamber spring 411 and the second chamber spring 421 are stretched, the inlet valves 171 and 172 are opened. On the other hand, the first separator 51 and the second separator 52 are also moved downward such that the volume of the cavity a and the cavity b becomes larger and the pressure becomes smaller. The first solution in the first chamber A and the second solution in the second chamber B enter the cavity a and the cavity b through the outlet ends of the fluid-sucking portion 211 and the fluid-sucking portion 212, respectively.

Spring reset: during the gradual reset of the spring, on one hand, when the first chamber spring 411 and the second chamber spring 421 are reset, the inlet valves 171 and 172 are closed. On the other hand, the first separator 51 and the second separator 52 are simultaneously moved upward such that the volume of the cavity a and the cavity b becomes smaller and the pressure becomes larger. Accordingly, the liquid that enters the cavity a and the cavity b flows out from the pump-out pipeline f1 and the pump-out pipeline f2 via the liquid pump outlet 61 of the first temporary reservoir cavity and the liquid pump outlet 62 of the second temporary reservoir cavity, respectively, thereby achieving the purpose of simultaneously delivering different solutions at the same time.

In the present embodiment, besides the portions of the pump-out pipeline f1 and the pump-out pipeline f2 in the press cover e are two separate delivery pipelines, there are the following designs:

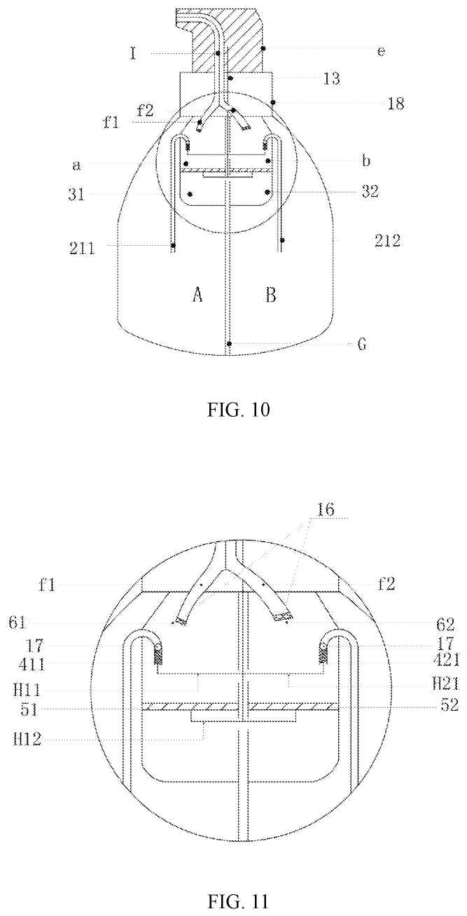

(1) The pump-out pipeline f1 and the pump-out pipeline f2 are disposed in the same delivery pipelines. When the separator I is disposed in one delivery pipeline, two delivery channels can be formed. The two delivery channels communicate with the pump-out pipeline f1 located in the first chamber A and the pump-out pipeline f2 located in the second chamber B, respectively (as shown in FIGS. 10, 11 and 12(1)).

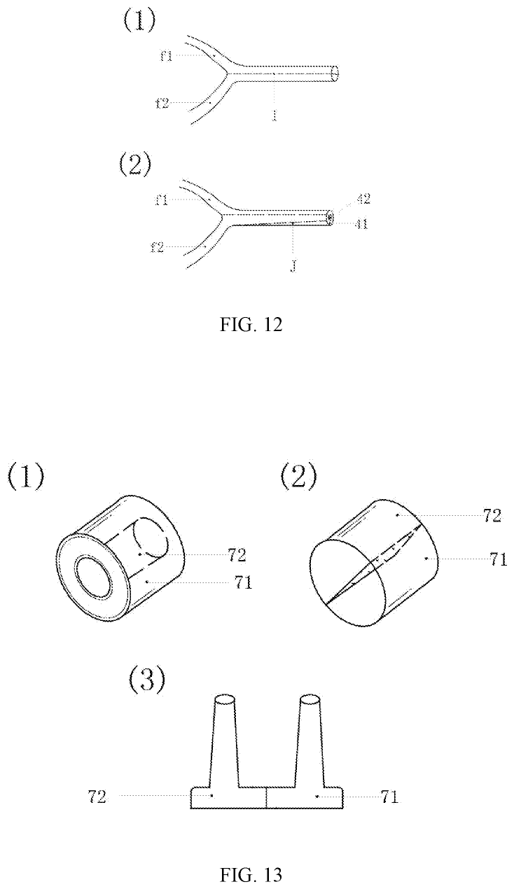

(2) When the portions of the pump-out pipeline f1 and the pump-out pipeline f2 in the press cover e are disposed in the same delivery pipeline, a round of separator J may be disposed in the delivery pipeline. The arrangement of a round of separator divides the cross section of the delivery pipeline into two concentric circles with different sizes such that the first solution in the first chamber A flows out from the outer side 41 of the delivery pipeline, while the second solution in the second chamber B flows out from the inner side 42 of the delivery pipeline. The exemplary specific arrangement of the pump-out pipeline f1 and the pump-out pipeline f2 is shown in FIG. 12(2).

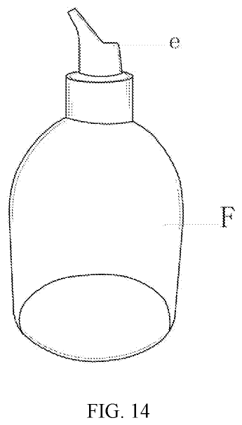

Certainly, when the portions of the pump-out pipeline f1 and the pump-out pipeline f2 in the press cover e are two separate delivery pipelines, a functional nozzle can be installed at the outlet of the pump-out pipeline f1 and the pump-out pipeline f2 on the press cover e to achieve the same delivery function as described above for design (1) and design (2). The exemplary structure of the nozzle is as shown in FIG. 13(3). After the nozzle with the structure shown in FIG. 13(3) is installed, the pump-out pipeline f1 enters the first liquid outlet 71 of the spray-head and the pump-out pipeline f2 enters the second liquid outlet 72 of the spray-head, thereby achieving the purpose of simultaneously delivering different solutions at the same time.

The dual-chamber drug delivery device as described above can be extended to a three-chamber, four-chamber, or even more chamber drug delivery device.

(3) The pump-out pipeline f1 and the pump-out pipeline f2 are pipelines with the same length but different cross-sectional areas (as shown in FIG. 2(2)). Therefore, after one press, the time during which first solution in the first chamber A and the second solution in the second chamber B flow out of the press device is different, thereby achieving the purpose of delivering different solutions one after another after one press.

(4) The pump-out pipeline f1 and the pump-out pipeline f2 are pipelines with the same cross-sectional area but different lengths (as shown in FIG. 2(3), for example, one is a linear pipeline and the other is a spirally curved pipeline). Therefore, after one press, the time during which the first solution in the first chamber A and the second solution in the second chamber B flow out of the press device is different, thereby also achieving the purpose of delivering different solutions one after another after one press.

In some embodiments, when the drug delivery device is used in a place where the administration site is small or where the administration site may be in contact with a small area (for example, at the inner wall of the nasal cavity, at the nasal limen or in the oral cavity), the shape of the press cover e can be as shown in FIG. 14.

In some embodiments, if it is desired to obtain a hazy solution, a device for atomizing the solutions delivered respectively from the pump-out pipeline f1 and the pump-out pipeline f2 can be provided outside the liquid outlets of the above-described drug delivery device.

The above dual-chamber drug delivery device can be used uprightly or used upside down.

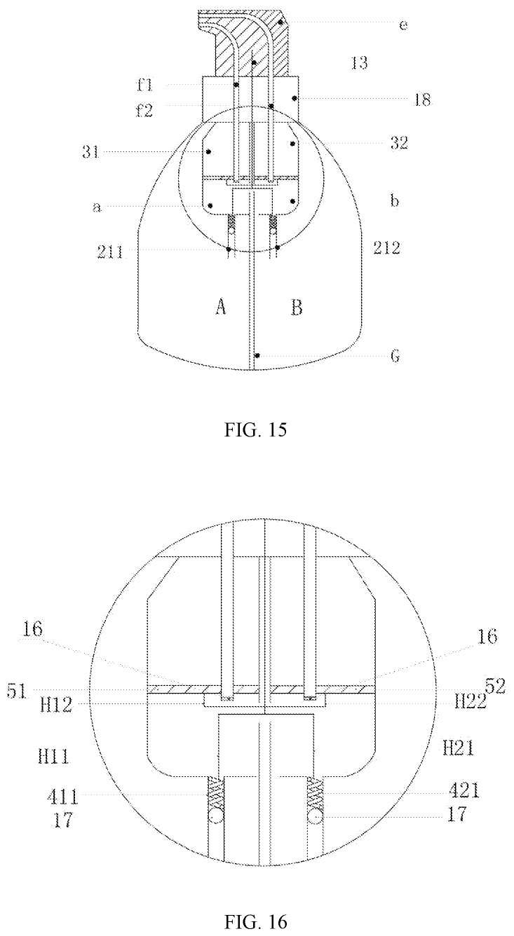

Referring to FIGS. 15 and 16, there is provided a drug delivery device capable of delivering two different solutions simultaneously, of which the structure is substantially identical to that of the drug delivery device as described in the preceding embodiments. The differences reside in that:

In the present embodiment, the outlet ends of the fluid-sucking portion 211 and the fluid-sucking portion 212 are located in a part of the cavity away from the press cover e, respectively (for the sake of clarity, the bottle mouth of the body is upward. The outlet ends of the fluid-sucking portion 211 and the fluid-sucking portion 212 are located below the the first separator 51 and the second separator 52, respectively, wherein the lower portion of the first separator 51 and the second separator 52 are the cavity a and the cavity b, respectively). The working principle of the drug delivery device is as follows: the press cover e is moved downward by the external pressure such that the gland connecting shaft 13 is simultaneously moved downward. As the connecting legs of the gland connecting shaft 13 are connected with the spring and the separator, respectively, in each chamber, when the gland connecting shaft 13 moves downward, the spring is compressed. On one hand, when the first chamber spring 411 and the second chamber spring 421 are compressed, the inlet valves 171 and 172 are closed. On the other hand, the first separator 51 and the second separator 52 are also moved downward such that the volume of the cavity a and the cavity b becomes smaller and the pressure becomes larger. The liquid stored in the cavity a and the cavity b flows out from the pump-out pipeline f1 and the pump-out pipeline f2 via the liquid pump outlet 61 of the first temporary reservoir cavity and the liquid pump outlet 62 of the second temporary reservoir cavity, respectively, thereby achieving the purpose of simultaneously delivering different solutions at the same time.

Spring reset: during the gradual reset of the spring, on one hand, the inlet valves 171 and 172 of the two chambers are opened, on the other hand, the first separator 51 and the second separator 52 are simultaneously moved upward such that the volume of the cavity a and the cavity b becomes larger and the pressure becomes smaller. The first solution in the first chamber A and the second solution in the second chamber B enters the cavity a and the cavity b via the outlet ends of the fluid-sucking portion 211 and the fluid-sucking portion 212, respectively, to wait for the next press.

That is, the drug delivery device of the present embodiment is capable of achieving simultaneously delivering two different solutions after one press.

In the present embodiment, besides the portions of the pump-out pipeline f1 and the pump-out pipeline f2 in the press cover e are two separate delivery pipelines, there are the following designs:

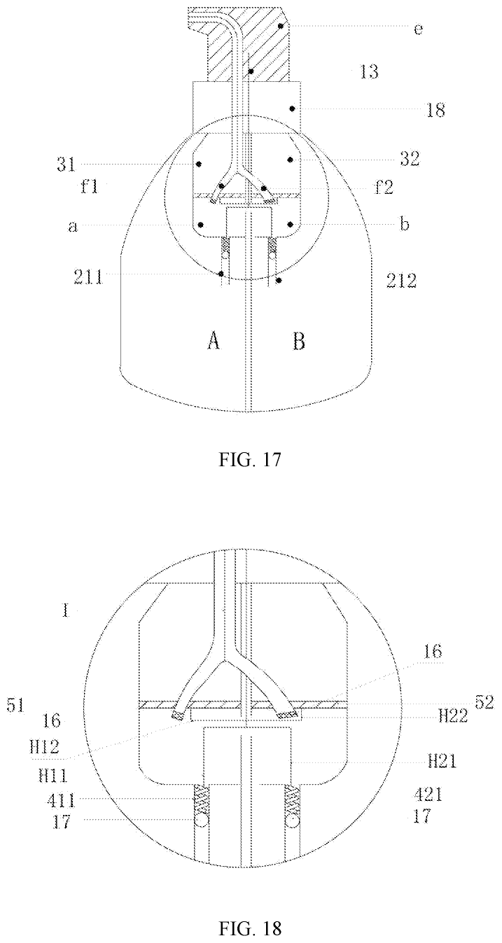

(1) The pump-out pipeline f1 and the pump-out pipeline f2 are disposed in the same delivery pipelines. When the separator I is disposed in one delivery pipeline, two delivery channels can be formed. The two delivery channels communicate with the pump-out pipeline f1 located in the first chamber A and the pump-out pipeline f2 located in the second chamber B, respectively (as shown in FIGS. 17 and 18).

(2) When the portions of the pump-out pipeline f1 and the pump-out pipeline f2 in the press cover e are disposed in the same delivery pipeline, a round of separator J may be disposed in the delivery pipeline. The arrangement of around of separator divides the cross section of the delivery pipeline into two concentric circles with different sizes such that the first solution in the first chamber A flows out from the outer side 41 of the delivery pipeline, while the second solution in the second chamber B flows out from the inner side 42 of the delivery pipeline. The exemplary specific arrangement of the pump-out pipeline f1 and the pump-out pipeline f2 is shown in FIG. 12(2).

Certainly, when the portions of the pump-out pipeline f1 and the pump-out pipeline f2 in the press cover e are in the two separate delivery pipelines, a functional nozzle can be installed at the outlet of the pump-out pipeline f1 and the pump-out pipeline f2 on the press cover e to achieve the same delivery function as described above for design (1) and design (2). The exemplary structure of the nozzle is shown in FIG. 13(3). After the nozzle with the structure shown in FIG. 13(3) is installed, the pump-out pipeline f1 enters the first liquid outlet 71 of the spray-head and the pump-out pipeline f2 enters the second liquid outlet 72 of the spray-head, thereby achieving the purpose of simultaneously delivering different solutions at the same time.

(3) The pump-out pipeline f1 and the pump-out pipeline f2 are pipelines with the same length but different cross-sectional areas (as shown in FIG. 2(2)). Therefore, after one press, the time during which first solution in the first chamber A and the second solution in the second chamber B flow out of the press device is different, thereby achieving the purpose of delivering different solutions one after another after one press.

(4) The pump-out pipeline f1 and the pump-out pipeline f2 are pipelines with the same cross-sectional area but different lengths (as shown in FIG. 2(3), for example, one is a linear pipeline and the other is a spirally curved pipeline). Therefore, after one press, the time during which the first solution in the first chamber A and the second solution in the second chamber B flow out of the press device is different, thereby also achieving the purpose of delivering different solutions one after another after one press.

The dual-chamber drug delivery device as described above can be extended to a three-chamber, four-chamber, or even more chamber drug delivery device.

In some embodiments, when the drug delivery device is used in a place where the administration site is small or where the administration site may be in contact with a small area (for example, at the inner wall of the nasal cavity, at the nasal limen or in the oral cavity), the shape of the press cover e can be shown in FIG. 14.

In some embodiments, if it is desired to obtain a hazy solution, a device for atomizing the solutions delivered respectively from the pump-out pipeline f1 and the pump-out pipeline f2 can be provided outside the liquid outlets of the above-described drug delivery device.

The above dual-chamber drug delivery device can be used uprightly or used upside down.

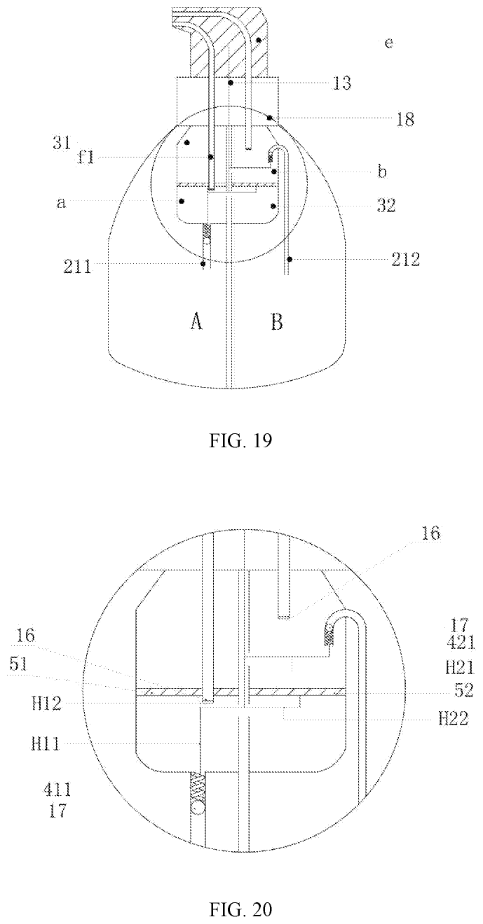

Referring to FIGS. 19 and 20, there is provided a drug delivery device capable of delivering two different solutions simultaneously, of which the structure is substantially identical to that of the drug delivery device as described in the preceding embodiments. The differences reside in that:

In the present embodiment, the outlet end of the fluid-sucking portion 211 is located in a part of the cavity away from the press cover e and the outlet end of the fluid-sucking portion 212 is located in a part of the cavity close to the press cover e (for the sake of clarity, the bottle mouth of the body is upward. The outlet end of the fluid-sucking portion 211 is located below the the first separator 51 and the outlet end of the fluid-sucking portion 212 is located above the second separator 52. The lower portion of the first separator 51 is the cavity a and the upper portion of the second separator 52 is the cavity b). The working principle of the drug delivery device is as follows: the press cover e is moved downward by the external pressure such that the gland connecting shaft 13 is simultaneously moved downward. As the connecting legs of the gland connecting shaft 13 are connected with the spring and the separator, respectively, in each chamber, when the gland connecting shaft 13 moves downward, the first chamber spring 411 is compressed, thereby closing the inlet valve 17 of the first spring device. At the same time, the first separator 51 is moved downward such that the volume of the cavity a becomes smaller and the pressure becomes larger. The liquid stored in the cavity a flows out from the pump-out pipeline f1 via the liquid pump outlet 61 of the first temporary reservoir cavity. On the other hand, the second chamber spring 421 is stretched to open the inlet valve 17 of the second spring device 42. At the same time, the second separator 52 is moved downward such that the volume of the cavity b becomes larger and the pressure becomes smaller. The second solution in the second chamber B enters the cavity b via the fluid-sucking portion 212 (i.e., when the cover e is pressed downward, only the first solution in the first chamber A is pumped out).

Spring reset: during the gradual reset of the spring, on one hand, the first chamber spring 411 is reset, the inlet valve 17 of the first spring device is opened. The first separator 51 is simultaneously moved upward such that the volume of the cavity a becomes larger and the pressure becomes smaller. The first solution in the first chamber A enters the cavity a via the fluid-sucking portion 211 to wait for the next press. On the other hand, the first chamber spring 421 is reset, the inlet valve 17 of the second spring device is closed. The second separator 52 is simultaneously moved upward such that the volume of the cavity b becomes smaller and the pressure becomes larger. The liquid stored in the cavity b flows out from the pump-out pipeline f2 via the liquid pump outlet 62 of the second temporary reservoir cavity (i.e., when the pressure is reset, only the second solution in the second chamber B is pumped out), thereby achieving the purpose of simultaneously delivering two different solutions after one press.

In the present embodiment, besides the portions of the pump-out pipeline f1 and the pump-out pipeline f2 in the press cover e are two separate delivery pipelines, there are the following designs:

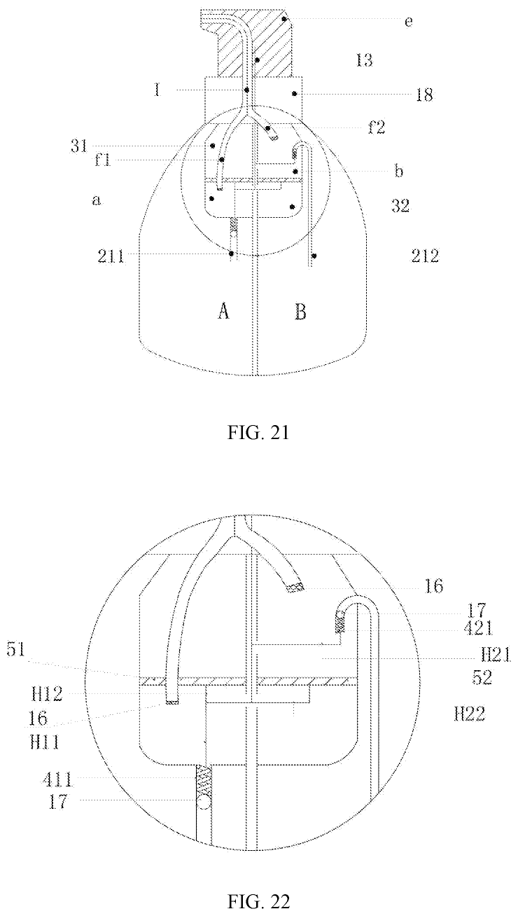

(1) The pump-out pipeline f1 and the pump-out pipeline f2 are disposed in the same delivery pipelines. When the separator I is disposed in a delivery pipeline, two delivery channels can be formed. The two delivery channels communicate with the pump-out pipeline f1 located in the first chamber A and the pump-out pipeline f2 located in the second chamber B, respectively (as shown in FIGS. 21 and 22).

(2) When the portions of the pump-out pipeline f1 and the pump-out pipeline f2 in the press cover e are disposed in the same delivery pipeline, a round of separator J may be disposed in the delivery pipeline. The arrangement of a round of separator divides the cross section of the delivery pipeline into two concentric circles with different sizes such that the first solution in the first chamber A flows out from the outer side 41 of the delivery pipeline, while the second solution in the second chamber B flows out from the inner side 42 of the delivery pipeline. The exemplary specific arrangement of the pump-out pipeline f1 and the pump-out pipeline f2 is shown in FIG. 12(2).

Certainly, when the portions of the pump-out pipeline f1 and the pump-out pipeline f2 in the press cover e are two separate delivery pipelines, a functional nozzle can be installed at the outlets of the pump-out pipeline f1 and the pump-out pipeline f2 on the press cover e to achieve the same delivery function as described above for design (1) and design (2). The exemplary structure of the nozzle is shown in FIG. 13(3). After the nozzle with the structure shown in FIG. 13 is installed, the pump-out pipeline f1 enters the first liquid outlet 71 of the spray-head and the pump-out pipeline f2 enters the second liquid outlet 72 of the spray-head, thereby achieving the purpose of simultaneously delivering different solutions at the same time.

(3) The pump-out pipeline f1 and the pump-out pipeline f2 are pipelines with the same length but different cross-sectional areas (as shown in FIG. 2(2)). Therefore, after one press, the time during which first solution in the first chamber A and the second solution in the second chamber B flow out of the press device is different, thereby achieving the purpose of delivering different solutions one after another after one press.

(4) The pump-out pipeline f1 and the pump-out pipeline f2 are pipelines with the same cross-sectional area but different lengths (as shown in FIG. 2(3), for example, one is a linear pipeline and the other is a spirally curved pipeline). Therefore, after one press, the time during which the first solution in the first chamber A and the second solution in the second chamber B flow out of the press device is different, thereby also achieving the purpose of delivering different solutions one after another after one press.

The dual-chamber drug delivery device as described above can be extended to a three-chamber, four-chamber, or even more chamber drug delivery device.

In some embodiments, when the drug delivery device is used in a place where the administration site is small or where the administration site may be in contact with a small area (for example, at the inner wall of the nasal cavity, at the nasal limen or in the oral cavity), the shape of the press cover e can be shown in FIG. 14.

In some embodiments, if it is desired to obtain a hazy solution, a device for atomizing the solutions delivered respectively from the pump-out pipeline f1 and the pump-out pipeline f2 can be provided outside the liquid outlet of the above-described drug delivery device.

The above dual-chamber drug delivery device can be used uprightly or used upside down.

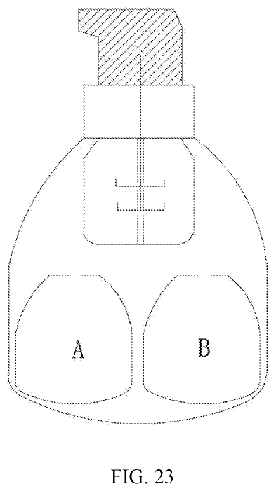

In the drug delivery devices of the foregoing embodiments, the first chamber A and the second chamber B can be two independent chambers disposed in the same container and separated by a hollow portion, or can be two separate containers that are not in contact or in contact with each other in the same container as the first chamber A and the second chamber B. Under the circumstances, the hollow portion is only used to separate the first temporary reservoir cavity 31 and the second temporary reservoir cavity 32 and there is no hollow portion between the first chamber A and the second chamber B (as shown in FIG. 23, the openings in the first chamber A and the second chamber B are used to accommodate the fluid-sucking portion 211 and the fluid-sucking portion 212, respectively).

In another aspect, the present disclosure relates a method for improving the stability of a drug comprising administering the drug to a subject with a drug delivery device, wherein the drug delivery device comprises:

a body for holding a first solution and a second solution which are independent with each other;

a pipeline delivery system comprising one or more pipelines for independently delivering the first solution and the second solution, respectively;

a press device for sucking the first solution and the second solution into the pipelines, and

a nozzle for delivering the first solution and the second solution in the pipelines to an administration site such that the first solution covers the second solution at the administration site.

In some embodiments, the drug is apomorphine or a pharmaceutically acceptable salt thereof.

In some embodiments, the pharmaceutically acceptable salt of apomorphine is selected from the group consisting of glucuronates, sulfates, phosphates, nitrates, hydrochlorides, dihydrochlorides, trihydrochlorides, hydrobromides, formates, acetates, besylates, methanesulfonates, ethanesulfonates, p-toluenesulfonates, phenpropionates, maleates, tartrates, fumarates, malates, ascorbates, citrates, mandelates, malonates, succinates, adipates, glycolates, lactates, aspartates, succinates, salicylates, acetyl salicylates, gallates, glutamates and sorbates.

In some embodiments, the first solution comprises the drug.

In some embodiments, the second solution comprises an alkaline polymer solution.

In the present disclosure, relational terms such as first, second and the like are used merely to distinguish one entity or operation from another entity or operation, and do not necessarily require or imply any such actual relationship or order between these entities or operations.

It will be understood from the foregoing that, although specific embodiments of the present disclosure have been described for purposes of illustration, various modification or improvements may be made by one skilled in the art without departing from the spirit and scope of the disclosure, and these variations or modifications are within the scope of the appended claims.

What is claimed is:

1. A drug delivery device, comprising
a body for holding a first solution and a second solution which are independent with each other;
a pipeline delivery system comprising one or more pipelines for independently delivering the first solution and the second solution, respectively;
a press device for sucking the first solution and the second solution into the pipelines, and
a nozzle for delivering the first solution and the second solution in the pipelines to an administration site such that the first solution covers the second solution at the administration site.

2. The drug delivery device of claim 1, wherein the body comprises a plurality of containers for independently holding the first solution and the second solution.

3. The drug delivery device of claim 1, wherein the body comprises a plurality of regions for independently holding the first solution and the second solution.

4. The drug delivery device of claim 1, wherein the pipelines comprise a suction portion and a delivery portion.

5. The drug delivery device of claim 4, wherein the suction portions of the pipelines are independent of each other.

6. The drug delivery device of claim 4, wherein the delivery portions of the pipelines are independent of each other.

7. The drug delivery device of claim 4, wherein the delivery portions of the pipelines are in contact with each other.

8. The drug delivery device of claim 1, wherein the press device comprises a pump and a tube.

9. The drug delivery device of claim 8, wherein the first solution and the second solution reach the administration site successively from the nozzles or the first solution and the second solution reach the administration site simultaneously.

10. The drug delivery device of claim 1, wherein the nozzle has one or more channels.

11. The drug delivery device of claim 1, wherein the nozzle has a first channel and a second channel, and the first channel and the second channel are independent with each other.

12. The drug delivery device of claim 1, wherein the nozzle has a first channel and a second channel, and the first channel is concentric with the second channel, preferably sections of the first channel and the second channels are all circular.

13. The drug delivery device of claim 1, wherein the nozzle has a first channel and a second channel, the first channel is located in the second channel, and cross-sectional release area of the first channel is greater than cross-sectional release area of the second channel.

14. The drug delivery device of claim 1, wherein the nozzle has a first channel and a second channel, the length of the first channel is identical to that of the second channel.

15. A method for improving stability of a drug, comprising administering the drug to a subject with the drug delivery device of claim 1.

16. The method of claim 15, wherein the drug is apomorphine or a pharmaceutically acceptable salt thereof.

17. The method of claim 15, wherein the first solution comprises the drug.

18. The method of claim 15, wherein the second solution comprises an alkaline polymer solution.
